(19) United States
(12) Patent Application Publication
(10) Pub. No.: US 2023/0180739 A1
(43) Pub. Date: Jun. 15, 2023

(54) NEW SWIM UP METHOD FOR SPERM WASHING

(71) Applicants: Michael AWADALLA, Cincinnati, OH (US); Ali AHMADY, Cincinnati, OH (US)

(72) Inventors: Michael AWADALLA, Cincinnati, OH (US); Ali AHMADY, Cincinnati, OH (US)

(21) Appl. No.: 18/065,788

(22) Filed: Dec. 14, 2022

Related U.S. Application Data
(63) Continuation of application No. 63/289,179, filed on Dec. 14, 2021.

(51) Int. Cl. A01N 1/02; C12N 5/076

(52) U.S. Cl. CPC A01N 1/0215; C12N 5/061; A01N 1/0284; A61D 19/025

(57) ABSTRACT

A sperm wash device for collecting motile sperm includes a first chamber section for holding an unwashed semen sample, a second chamber section for holding sperm wash media disposed over and attached to the first chamber section, and a ledge separating the first chamber section from the second chamber section. The ledge includes a slot therein that is configured be filled with sperm wash media that does not get recovered. A slider is placed on the ledge. Characteristically, the slider is moveable between a first position and a second position. At the first position, the slider allows fluid communication between the first chamber section and the second chamber section. At the second position, the slider blocks fluid communication between the first chamber section and the second chamber section. A method of using the sperm wash device is also provided.

### CROSS-REFERENCE TO RELATED APPLICATIONS

This application claims the benefit of U.S. provisional application Ser. No. 63/289,179 filed Dec. 14, 2021, the disclosure of which is hereby incorporated in its entirety by reference herein.

### TECHNICAL FIELD

In at least one aspect, a device for washing and collecting semen is provided.

### BACKGROUND

Sperm washing is required to prepare sperm for use with in vitro fertilization (IVF) and intrauterine insemination (IUI). Washing semen removes dead sperm, immotile sperm, leukocytes, epithelial cells, and other cellular debris, which are thought to be harmful to viable sperm (1,2). Washing also allows for a suspension of sperm in an appropriate volume of fluid at an appropriate concentration for its intended use. With assisted reproduction, the three main use scenarios are IUI, IVF, and IVF with ICSI. Current methods of semen washing and preparation for assisted reproduction are sub-optimal due to the embryologist's time commitment required for pipetting, use of a centrifuge which can damage sperm, and sub-optimal recovery of motile sperm.

Density gradient centrifugation and swim-up are the two most common methods used for sperm washing for assisted reproduction. These methods are associated with high pregnancy rates compared to other methods after intrauterine insemination (3). Studies have shown density gradient to be associated with better post-wash morphology and higher live birth rates with IVF than swim up (4,5). A simple wash can also be used but is not commonly performed in modern practice mainly because it removes the least cellular debris from the motile sperm. These commonly used methods all have multiple pipetting and centrifugation steps.

There is concern that multiple centrifugation steps involved in these methods may damage sperm through reactive oxygen species, which is thought to increase DNA fragmentation (6-8). The proposed mechanism of action is that the centrifugation causes sperm to form a pellet with other cells in the ejaculate (9). A recent review paper concluded that sperm separation methods should be quick, easy, low cost, and ideally avoid centrifugation (9). Another review article recommends investigational methods focus on processing raw semen samples rather than prewashed samples (10).

A newer method of sperm separation uses microfluidic chips. Use of microfluidic chips does not require a centrifuge and requires less andrologist hands-on time for use. While microfluidic chips have shown a lower sperm DNA fragmentation index after processing than density gradient centrifugation, there is limited data on live birth rate comparisons (11,12). A systematic review and meta-analysis found low DNA fragmentation to be associated with live birth after IVF and ICSI but no difference in live birth rates in the subgroup of patients treated with ICSI (13). Another systematic review found DNA fragmentation to be associated with pregnancy after IVF (OR 1.65) and a weaker association with pregnancy after ICSI with an OR of 1.31 (14). This suggests that in patients with high DNA fragmentation ICSI alone might be helpful. It is uncertain if sperm washing techniques that are associated with low post wash DNA fragmentation impact live birth rates or if live birth rates are simply related to pre wash DNA fragmentation.

There are four drawbacks to use of microfluidic chips. First, there is limited data on live birth outcomes when sperm is processed with a microfluidic device compared to other methods. Second, the cost of the device can range from $100-200 and adds to the total cost of IVF. Third, most microfluidic devices are only capable of processing limited volumes of semen and some are specifically designed for use with small volumes (15). If the volume exceeds the capacity of a device additional devices need to be used or else the excess volume cannot be utilized. Lastly, while the devices recover good-quality sperm (as measured by morphology and DNA fragmentation) the quantity (concentration and total motile count) of sperm recovered is often low (16).

### SUMMARY

In at least one aspect, a sperm wash device for collecting motile sperm is provided. The sperm wash device includes a first chamber section for holding an unwashed semen sample, a second chamber section for holding sperm wash media disposed over and attached to the first chamber section, and a ledge separating the first chamber section from the second chamber section. The ledge includes a slot therein that is configured to be filled with sperm wash media that does not get recovered. A slider is placed on the ledge. Characteristically, the slider is moveable between a first position and a second position. At the first position, the slider allows fluid communication between the first chamber section and the second chamber section. At the second position, the slider blocks fluid communication between the first chamber section and the second chamber section. Advantageously, during operation, the unwashed semen sample is placed in the first chamber section with the slider at the first position, and then the slider is positioned in the second position, the sperm wash media is introduced into the second chamber section and after the sperm wash media is added, the slider is repositioned in the first position thereby allowing sperm to travel from the first chamber section to the second chamber section.

In another aspect, a method for collecting motile sperm with the sperm wash device set forth herein is provided. The method includes steps of:
a) introducing the unwashed semen sample into the first chamber section while the slider is in the first position;
b) moving the slider to the second position;
c) introducing the sperm wash media into the second chamber section;
d) positioning the slider to the first position;
e) allow sperm to swim from the first chamber section to the second chamber section for a predetermined period of time;
f) positioning the slider in the second position; and
g) collecting the sperm wash media having motile sperm therein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIGS. 1A and 1B. Cross sections of the chamber sections of a sperm wash device.

FIGS. 1C-1 and 1C-2. 3D models of slider device.

FIG. 2. Perceptive view of a slider used in the sperm wash device.

FIGS. 3A and 3B. (A) 3D model and (B) 2D drawing of the device with one side cut away showing chamber for liquefied semen sample at the bottom and sperm wash media at the top. An L shaped cutout is seen in the top section to allow for insertion or removal of the barrier between the two compartments.

FIG. 4. % TMC recovery at 30 minutes comparing room temperature to 37° C. with split samples (n=3).

FIG. 5. % TMC recovery at 15, 30, and 60 minutes at 37° C. with split samples (n=3).

FIG. 6. % TMC recovery with density gradient compared to 30 minutes in the device at 37° C. with split samples (n=10).

FIG. 7. % normal morphology comparing density gradient compared to 30 minutes in the device at 37° C. with split samples. Data for this figure used the same samples as FIG. 6 with the exception that two samples had too few sperm for morphology analysis with the device so the final n is 8.

FIG. 8. 3D printed prototype devices for processing 1 mL (left) and 2 mL (right) of semen.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B”. In the ease of “only A”, the term also covers the possibility that B is absent, i.e. “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset. In a refinement, “one or more” includes “two or more.”

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numeral quantity, in a refinement, the term “less than” includes a lower non-included limit that is 5 percent of the number indicated after “less than.” For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, or 1 percent of the number indicated after “less than.”

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

Abbreviations:

“FDM” means fused deposition modeling.

“HIPS” means high-impact polystyrene.

“IUI” means intrauterine insemination.

“IVF” means in vitro fertilization.

“MHM” means Multipurpose Handling Media.

“TMC” means total motile count.

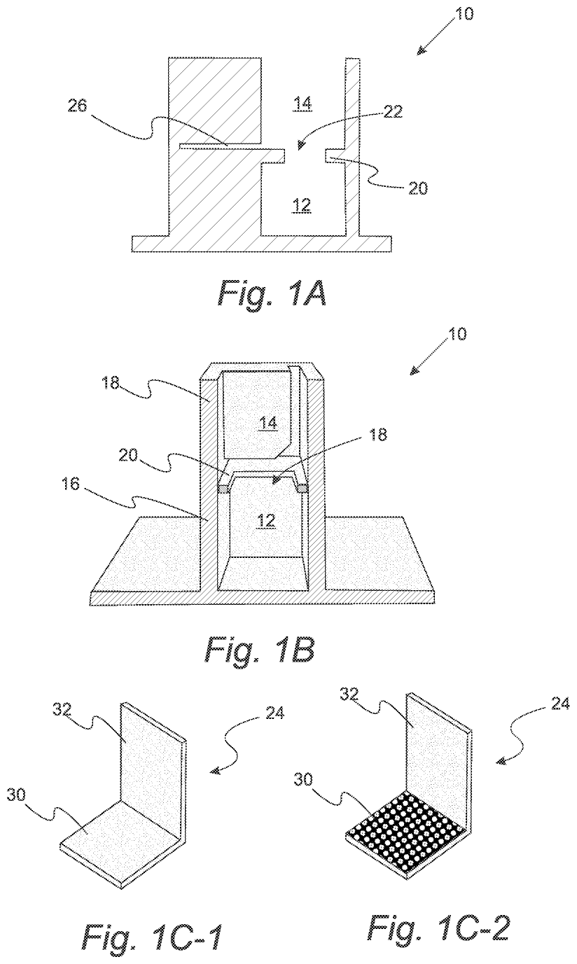

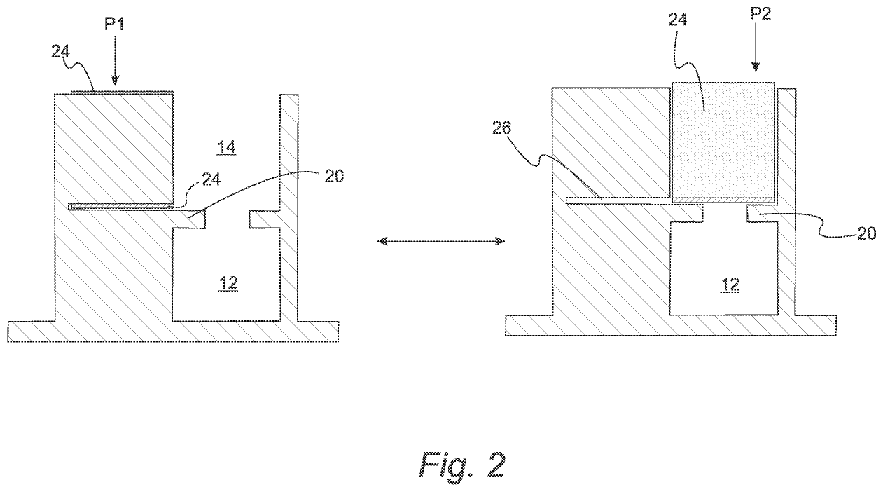

Referring to FIGS. 1A, 1B, 1C-1, and 1C-2, a sperm wash device is schematically illustrated. Sperm washing device 10 includes a first chamber section 12 for holding an unwashed semen sample and a second chamber section 14 for holding sperm wash media disposed over and attached to (e.g., in fluid communication with) the first chamber section 12. Typically, first chamber section 12 includes a first plurality of sidewalls 16 (e.g., 4 sidewalls) that defines a first cavity for holding the unwashed semen sample and second chamber section 14 includes a second plurality of sidewalls 18 (e.g., 4 sidewalls) that define a first cavity for holding the sperm wash media. Typically, the first chamber section 12 includes a first cavity having a volume from about 0.2 ml to 3 ml and the second chamber section 14 includes a second cavity having a volume from about 0.2 ml to 3 ml. In some refinements, the first chamber section 12 includes a first cavity having a volume of at least 0.2 ml, 0.3 ml, 0.5 ml, 0.8 ml, 1 ml, 1.2 ml, 1.5 ml, or 1.8 ml and a volume of at most 4 ml, 3.5 ml, 3 ml, 2.5 ml, 2.2 ml, 2 ml, 1.8 ml, or 1.5 ml. In some refinements, the second chamber section 14 includes a second cavity having a volume of at least 0.2 ml, 0.3 ml, 0.5 ml, 0.8 ml, 1 ml, 1.2 ml, 1.5 ml, or 1.8 ml and a volume of at most 4 ml, 3.5 ml, 3 ml, 2.5 ml, 2.2 ml, 2 ml, 1.8 ml, or 1.5 ml.

Still referring to FIGS. 1A, 1B, 1C-1, and 1C-2, ledge 20 separates the first chamber section 12 from the second chamber section 14. The ledge 20 defines an opening 22 (i.e., a slot) therein that is configured to be filled with sperm wash media that does not get recovered. During operation, slider 24 is placed on the ledge 20. Slider 24 includes a bottom section 30 attached to a side section 32. Typically, bottom section 30 and side section 32 are planar sections (i.e., flat). In a refinement, slider 24 can have an “L shape” where side section is substantially perpendicular to bottom section 30. The bottom section 30 is configured to reversibly cover opening 22 as explained below. FIG. 1C-1 depicts a variation in which bottom section 30 is composed of a solid material (e.g., non-mesh or non-porous material), while FIG. 1C-2 depicts a variation in which bottom section 30 is composed of a mesh. In a refinement, such a mesh includes openings having a diameter from about 20 microns to 100 microns.

FIG. 2 depicts the operation of slider 24. Slider 24 is moveable between a first position P1 and a second position P2. In this regard, second chamber section 14 includes a slit 26 that is adapted to receive slider 24 when it is positioned at first position P1. At the first position P1, slider 24 allows fluid communication between the first chamber section 12 and the second chamber section 14. At the second position, slider 24 blocks fluid communication between the first chamber section 12 and the second chamber section 14. In particular, the bottom section 30 of slider 24 is configured to cover opening 22 when the slider is positioned at the second position. During operation, the unwashed semen sample is placed in the first chamber section 12 while slider 24 is at the first position P1. Slider 24 is then positioned in the second position P2. With the slider in the second position P2, the sperm wash media is introduced into the second chamber section. After the sperm wash media is added, slider 24 is repositioned in the first position thereby allowing sperm to travel from the first chamber section to the second chamber section.

In a variation, first chamber section 12 and second chamber section 14 are independently composed of a plastic. Examples of useful plastics include, but are not limited to, high impact polystyrene, polystyrene, polyolefins, nylon, and combinations thereof. In a refinement, the plastic is 3D printable or injection moldable.

In another embodiment, a method for collecting motile sperm with the sperm wash device of FIGS. 1A, 1B, 1C-1, and 1C-2. The method includes steps of:
a) introducing the unwashed semen sample into the first chamber section while the slider is in the first position;
b) moving the slider to the second position;
c) introducing the sperm wash media into the second chamber section;
d) positioning the slider to the first position;
e) allow sperm to swim from the first chamber section to the second chamber section for a predetermined period of time;
f) positioning the slider in the second position; and
g) collecting the sperm wash media having motile sperm therein.

Typically, the unwashed semen sample is liquified prior to being introduced into the first chamber section. In a refinement, the sperm wash device is heated during the collection of the motile sperm. In a further refinement, the sperm wash device is heated to a temperature from about 28 to 45° C. during collection of the motile sperm.

In a variation, the predetermined period of time during which sperm are collected is from about 10 to 90 minutes. In a refinement, the predetermined period of time is about 30 minutes. In some refinements, the predetermined period of time is at least 5 minutes, 10 minutes, 15 minutes, 20 minutes, 25 minutes, 30 minutes, 35 minutes, or 40 minutes. In further refinements, the predetermined period of time is at most 120 minutes, 110 minutes, 100 minutes, 90 minutes, 80 minutes, 70 minutes, 60 minutes, or 50 minutes.

The following examples illustrate the various embodiments of the present invention. Those skilled in the art will recognize many variations that are within the spirit of the present invention and the scope of the claims.

The aim of this study was to develop a new method for sperm washing and benchmark performance by comparing % total motile sperm count recovery and post-wash morphology with that of density gradient preparation. Specifically, a goal is to develop a new method that does not use centrifugation.

### Materials and Methods

### Study Population

This study enrolled male patients at least 18 years old having semen analysis at USC Fertility. Subjects were excluded if they were having IUI, sperm cryopreservation, or had any clinical use for the sperm after the semen analysis. Written informed consent was obtained from all participants. This study was approved by the University of Southern California Institutional Review Board (HS-19-00052).

### Device Manufacturing and Use

The sperm wash devices were manufactured with 3D printing using high-impact polystyrene (HIPS). The device has two components: the main device and an inserted slider. The main device has four parts. The first part is a 1 mL chamber for an unwashed semen sample. The second part is 2 mm of vertical space that fills with sperm wash media that does not get recovered. This is to decrease the chance of pipetting off unwashed semen. This space also contains a ledge for the slider to rest on. The third part is a 1.6 mm wide lateral slit for the 1.0 mm thick slider to slide into. The fourth part is a 1 mL chamber at the top to hold the sperm wash media that the sperm swims up into.

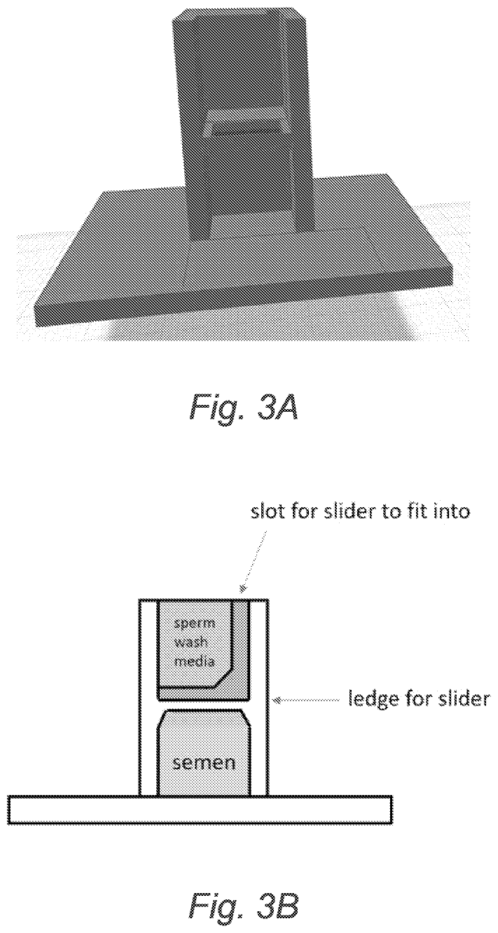

Use of the device begins with allowing the semen sample to liquify in the collection cup for 15 minutes in an incubator at 37° C. Washing steps were performed with sperm wash media consisting of Multipurpose Handling Media (MHM) with gentamicin supplemented with 12% Serum Substitute Supplement (Irvine Scientific, Santa Ana, Calif.). 1 mL of liquefied semen was placed in the bottom of the device (FIG. 3). A slider was placed over the liquified semen to prevent turbulent mixing of the semen and sperm wash media. 1 mL of sperm wash media was placed in the device and the slider was moved out of the way by sliding it laterally into the device slot and thus allowing contact between the semen and sperm wash media. Different swim up incubation times were tested and 30 minutes was used for the comparison to density gradient. After the allotted time for swim up the slider was slid back over the semen sample, and the sperm wash media containing sperm that swam up into the media was pipetted off starting from the top of the media (the media and air interface) and progressing further towards the bottom of the well. This minimizes the risk of pipetting any unwashed semen. A new device was used for each test and was discarded after single use.

### Density Gradient Centrifugation

Density gradient centrifugation was performed using a 90% density gradient (Isolate sperm separation medium stock solution: Irvine Scientific, Santa Ana, Calif.). Liquefied semen samples were diluted 1:1 with MHM and mixed well. Up to 2.5 mL of the diluted solution was overlayed on 2 mL of density gradient. The sample was then centrifuged at 300×g for 30 minutes. After centrifugation overlying seminal fluid and gradient were removed. The pellet was then resuspended in 4 mL of sperm wash media and then centrifuged at 400×g for 7 minutes. The supernatant was then removed, and the pellet was resuspended in approximately 0.5 mL of MHM. MicroCell counting chambers were used for sperm counts. All tests were performed using paired samples split into 1 mL portions.

### Results

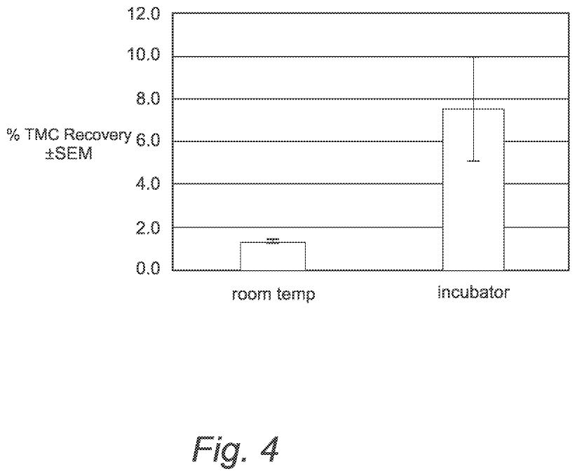

A first test of temperature showed that placing the device in the incubator during the time allotted for sperm swim up was essential to obtaining adequate recovery of the total motile sperm count. The mean % TMC recovery at 30 minutes was 1.3% at room temperature compared to 7.5% in the incubator at 37° C. (FIG. 4). This informed the decision to perform the subsequent tests with the device in the incubator.

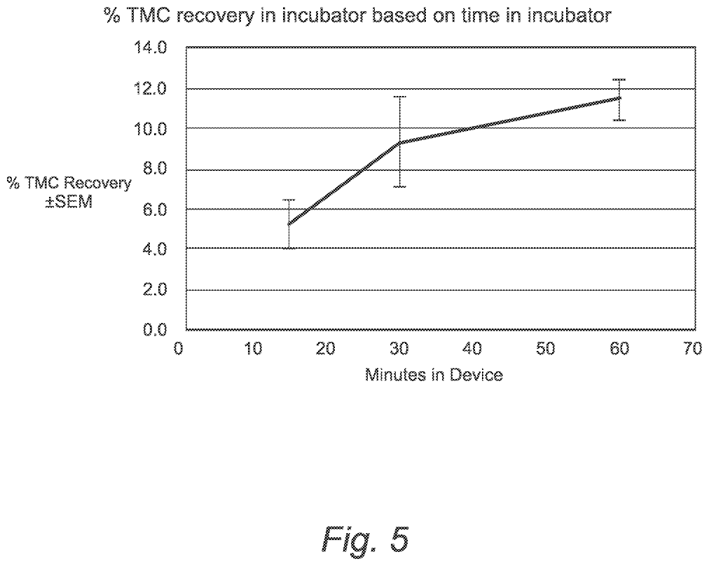

In a second test, it was found that longer times in the device were associated recovery of greater quantities of sperm. The % TMC recovery with the device in the incubator was 5.2% at 15 minutes, 9.3% at 30 minutes, and 11.5% at 60 minutes as shown in FIG. 5. The greatest increase in % TMC recovery was in going from 15 to 30 minutes. Although there was still some increase in recovery with additional incubation to 60 minutes, a decision was made to proceed with subsequent tests using 30 minutes as this balanced sperm recovery with the clinical need to perform a quick wash procedure.

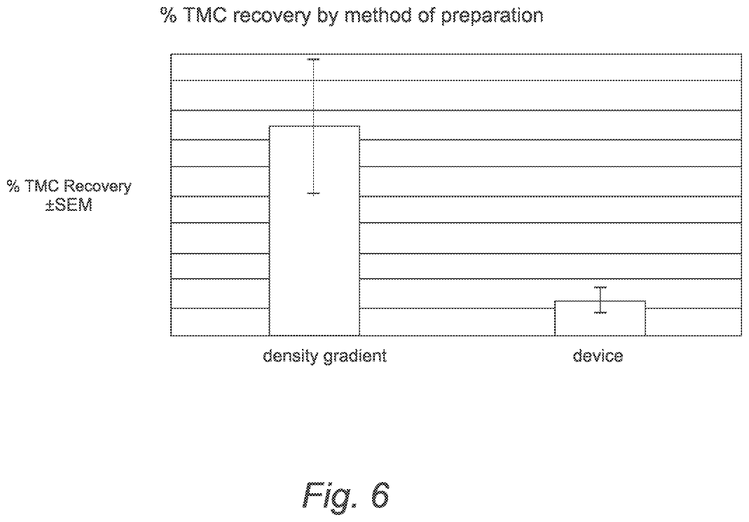

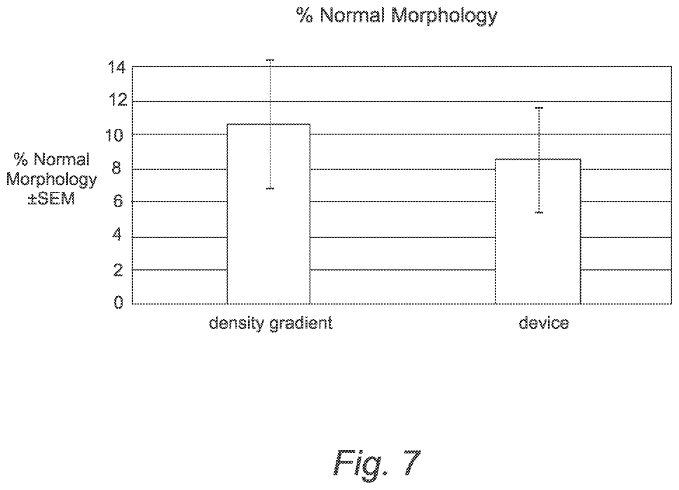

In a final analysis, the % TMC recovery using the device in the incubator for 30 minutes was 6.4% compared to 37.0% using density gradient, as shown in FIG. 6. The percentage of normal morphology using the device was 8.5% compared to 10.6% using density gradient, as shown in FIG. 7. To evaluate for sperm toxicity of HIPS, the TMC of 1 mL samples in the device after liquification and 60 minutes later at room temperature were compared. Compared to initial TMC the counts at 60 minutes were 104%, 93%, and 82% of the initial counts for 3 tested samples.

### Discussion

The method presented avoids two of the main limitations of commonly used microfluidic devices. First, the small microfluidic channels that the sperm swim through in a microfluidic device likely limit the quantity of sperm recovered. Long channels several centimeters in length with small cross-sectional areas of several square millimeters likely contribute to low numbers of high-quality sperm retrieved. The device presented here has a short channel length of 2 mm (minimum length required for sperm to swim up and be recovered) and a large cross-sectional area of 81 mm² (the chamber dimensions are 10×10×10 mm with a 1 mm ledge at the top). This allows for recovery of larger numbers of sperm. Likely larger numbers of sperm would mean lower sperm quality, but this decrease in quality may not have much clinical significance. The second design limitation of commonly used microfluidic devices is the size of the membrane pores that the sperm swim through. Based mainly on average sperm head diameters of approximately 5 μm, commonly used pore sizes that the sperm swim through are 8 μm to allow for some wiggle room (6). Small pore sizes likely limit sperm movement across the membrane and increase the incubation time needed for the recovery of sufficient amounts of sperm. A design modification that would likely allow for the recovery of greater quantities of sperm would be to set the pore size just below the diameter of other non-sperm cells. It is likely that a pore diameter of 50-100 μm would be optimal as this would be too small for any cells other than sperm to fit through especially given that sperm cells are the only highly motile cells in the ejaculate.

The new device-based method described here uses direct contact between the liquified semen and the overlaying wash media. This avoids the use of pores and likely allows for maximal sperm movement to facilitate swimming up into the wash media. However, prior to clinical use additional studies are needed to ensure that the recovered media is sufficiently free of other cellular debris and bacteria. If contamination is an issue with direct contact, a membrane with pores or a mesh (such as a cell strainer mesh) could be used at the interface of the liquefied semen and the overlying wash media. Incorporation of a membrane or mesh would need to be carefully designed to prevent air gaps in the device which would prevent sperm movement from the liquefied semen into the overlaid sperm wash media.

There are several important design considerations with this device and method. Essentially, this is a direct swim up method with the device used mainly to allow for controlled contact or separation between the two compartments (liquefied sperm and overlying sperm wash media). A critical aspect of the design is the slider which allows precise layering of the MHM on top of the liquefied semen sample. This is important because with precise layering the sperm can be drawn off the top and used directly without further centrifugation and resuspension. The slider is used to separate the compartments to avoid turbulence when the overlying sperm wash media is placed. The device allows the slider to slide out laterally to allow contact between the two layers. If the slider is removed vertically, this will create turbulent mixing between the wash media and the unwashed semen. This device could theoretically also work without a separate 15-minute liquification step, or the liquification and swim up steps could be combined by simply skipping the liquification step and increasing the swim up time from 30 to 45 minutes. Using unliquified semen is preferred as this would remove a step and speed up processing (10). However, sperm recovery may be lower with the use of an unliquified semen sample.

It was found that HIPS plastic is ideal for rapid prototyping with 3D printing. This allowed for an iterative design process that was essential for the development of the described method and device. Most early prototypes had a recovery of <1% of the TMC. HIPS is ideal for this application because it is relatively hydrophobic compared to other plastics and as with most plastics biologically inert. 3D printing with fused deposition modeling (FDM) uses a hot nozzle to melt the plastic for extrusion. This process produces a slightly porous plastic device. Hydrophobic plastic is needed to prevent leakage through the device. 3D printing with HIPS is also known to produce low levels of volatile organic compounds. HIPS is similar structurally to polystyrene, which most IVF labware is made of. One drawback of 3D printing with HIPS is that higher nozzle temperatures and print bed temperatures are needed than most other plastics commonly used with 3D printing. An enclosure is also needed to control the temperature and prevent layer separation due to differential cooling. While HIPS is a convenient plastic for rapid prototyping of models that work like the final intended device, devices for clinical use will likely be made from injection-molded polystyrene similarly to most labware used in IVF. Polystyrene is desired for laboratory use because it can be manufactured to produce a transparent product. Since high temperatures are needed to melt polystyrene, the addition of butadiene to form HIPS is needed to decrease the melting temperature and allow for 3D printing.

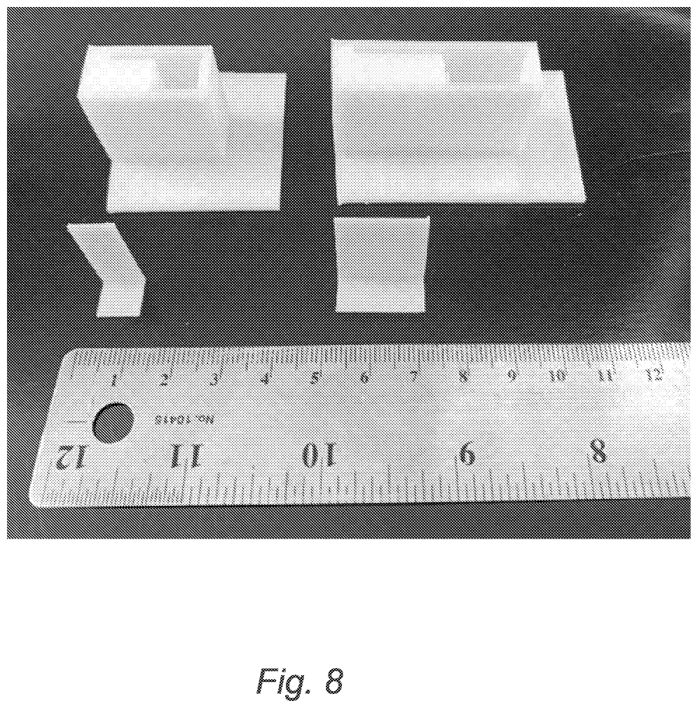

The design of this device can be easily modified to accommodate any volume of semen essentially by stretching out the dimensions. FIG. 8 shows a 1 mL device on the left and a 2 mL device on the right. In actual use the devices will likely need to be made in different sizes of 0.5 mL increments (1 mL, 1.5 mL, 2 mL, 2.5 mL, and 3 mL) to maximize sperm recovery with any volume of semen. With the 1 mL device, overlying 1 mL of sperm wash media is optimal as this allows for pipetting off approximately 0.4-0.6 mL of media which is appropriate for IUI, IVF, or IVF/ICSI. Some of the 1 mL sperm wash media is not recovered as it remains close to the unwashed semen sample or in the slot for the slider and is unrecoverable. With devices for larger semen volumes the optimal volume of sperm wash media will need to be determined to end up with approximately 0.4-0.6 mL of media pipetted off at the end.

There are some device improvements that likely will result in improved recovery of motile sperm. The comparison in FIG. 6 of total motile recovery shows 37.0% recovery of TMC with density gradient and only 6.4% recovery with the device. This comparison is meant to benchmark the performance of the device in its current state with the best available current practice. Density gradient centrifugation is a mature, optimized technology at this point, while the current device has not yet undergone optimization. The device can be manufactured with lower tolerances on the slider and slot to decrease the dead space where sperm wash media cannot be recovered from. In the current model, the slider is 1.0 mm thick and slides into a 1.6 mm slot. These tolerances are based on the limitations of FDM 3D printing. A swim up time of 60 minutes could be used instead of 30 minutes to help increase the amount of recovered sperm. More wash media could be used and a second step could be added to filter some of the sperm wash media to concentrate the sperm back down to an appropriate volume.

This device has multiple use scenarios. This would allow for sperm wash for IUI to be performed at the bedside, which will allow for a continuous chain of custody of the sperm sample to be verified by the male partner. This method would also likely work well for IUI at a general ob/gyn office, IUI in resource-poor settings, and for processing samples for IVF and IVF/ICSI. Processing samples with low sperm counts for IUI will likely not work well with this method unless significant improvements can be made; density gradient centrifugation will likely be a better option. This device is well suited for use in an automated andrology lab since the pipetting required is simple.

### Conclusions

Studies on sperm separation techniques are best performed on split samples to control for sample-to-sample variation. The device presented is a tool to allow for precise layering and pipetting. This method is able to recover sufficient sperm for IVF for most patients and for IUI in patients with good sperm counts. This device is one of the simplest methods proposed for sperm wash and as a result has expanded use scenarios. Specifically, this device can be used for everyday IUI and IVF, at the bedside, in resource poor settings, and can allow for an uninterrupted chain of custody of the sperm by the male patient.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Mortimer D. Sperm preparation techniques and iatrogenic failures of in-vitro fertilization. Hum. Reprod. 1991;6:173-6.

2. Fariello R M, Del Giudice P T, Spaine D M, Fraietta R, Bertolla R P, Cedenho A P. Effect of leukocytospermia and processing by discontinuous density gradient on sperm nuclear DNA fragmentation and mitochondrial activity. J Assist Reprod Genet 2009;26:151-7.

3. Carrell D T, Kuneck P H, Peterson C M, Hatasaka H H, Jones K P, Campbell B F. A randomized, prospective analysis of five sperm preparation techniques before intrauterine insemination of husband sperm. Fertil Steril 1998;69:122-6.

4. Prakash P, Leykin L, Chen Z, Toth T, Sayegh R, Schiff I, et al. Preparation by differential gradient centrifugation is better than swim-up in selecting sperm with normal morphology (strict criteria). Fertil Steril 1998;69:722-6.

5. Van Der Zwalmen P, Bertin-segal G, Geerts L, Debauche C, Schoysman R. Sperm morphology and IVF pregnancy rate: Comparison between percoll gradient centrifugation and swim-up procedures. Hum Reprod 1991;6:581-8.

6. Asghar W, Velasco V, Kingsley J L, Shoukat M S, Shafiee H, Anchan R M, et al. Selection of Functional Human Sperm with Higher DNA Integrity and Fewer Reactive Oxygen Species. Adv Healthc Mater 2014;3:1671-9.

7. Zini A, Finelli A, Phang D, Jarvi K. Influence of semen processing technique on human sperm DNA integrity. Urology 2000;56:1081-4.

8. Aitken J R, Clarkson J S. Significance of Reactive Oxygen Species and Antioxidants in Defining the Efficacy of Sperm Preparation Techniques. J Androl 1988;9:367-76.

9. Rappa K L, Rodriguez H F, Hakkarainen G C, Anchan R M, Mutter G L, Asghar W. Sperm processing for advanced reproductive technologies: Where are we today? Biotechnol. Adv. 2016;34:578-87.

10. Samuel R, Feng H, Jafek A, Despain D, Jenkins T, Gale B. Microfluidic-based sperm sorting & analysis for treatment of male infertility. Transl. Androl. Urol. 2018;7:S336-47.

11. Quinn M M, Jalalian L, Ribeiro S, Ona K, Demirci U, Cedars M I, et al. Microfluidic sorting selects sperm for clinical use with reduced DNA damage compared to density gradient centrifugation with swim-up in split semen samples. Hum Reprod 2018;33:1388-93.

12. Shirota K, Yotsumoto F, Itoh H, Obama H, Hidaka N, Nakajima K, et al. Separation efficiency of a microfluidic sperm sorter to minimize sperm DNA damage. Fertil Steril 2016;105:315-321.e1.

13. Osman A, Alsomait H, Seshadri S, El-Toukhy T, Khalaf Y. The effect of sperm DNA fragmentation on live birth rate after IVF or ICSI: A systematic review and meta-analysis. Reprod. Biomed. Online. 2015;30:120-7.

14. Simon L, Zini A, Dyachenko A, Ciampi A, Carrell D. A systematic review and meta-analysis to determine the effect of sperm DNA damage on in vitro fertilization and intracytoplasmic sperm injection outcome. Asian J. Androl. 2017;19:80-90.

15. Cho B S, Schuster T G, Zhu X, Chang D, Smith G D, Takayama S. Passively Driven Integrated Microfluidic System for Separation of Motile Sperm. Anal Chem 2003;75:1671-5.

16. Parrella A, Xie P, Keating D, Rosenwaks Z, Palermo Ronald O Perel-G D, Akcay B, et al. Microfluidic Selection Of Spermatozoa Retains Chromatin Integrity and Yields Higher Pregnancy Rates. 2018.

What is claimed is:

1. A sperm wash device comprising:
a first chamber section for holding an unwashed semen sample;
a second chamber section for holding sperm wash media disposed over and attached to the first chamber section;
a ledge separating the first chamber section from the second chamber section, the ledge having a slot therein that is configured be filled with sperm wash media that does not get recovered; and
a slider is placed on the ledge, the slider being moveable between a first position and a second position, wherein at the first position, the slider allows fluid communication between the first chamber section and the second chamber section and at the second position, the slider blocks fluid communication between the first chamber section and the second chamber section, and
wherein during operation, the unwashed semen sample is placed in the first chamber section with the slider at the first position and then the slider is positioned in the second position, the sperm wash media is introduced into the second chamber section, after the sperm wash media is added, the slider is repositioned in the first position thereby allowing sperm to travel from the first chamber section to the second chamber section.

2. The sperm wash device of claim 1, wherein the second chamber section includes a slit adapted to receive the slider when positioned at the first position.

3. The sperm wash device of claim 1, wherein the first chamber section and the second chamber section are composed of a plastic.

4. The sperm wash device of claim 3, wherein the plastic is selected from the group consisting of polystyrene, polyolefins, nylon, and combinations thereof.

5. The sperm wash device of claim 3, wherein the plastic is 3D printable.

6. The sperm wash device of claim 1, wherein the first chamber section includes a first plurality of sidewalls that define a first cavity for holding the unwashed semen sample.

7. The sperm wash device of claim 1, wherein the second chamber section includes a second plurality of sidewalls that define a first cavity for holding the sperm wash media.

8. The sperm wash device of claim 1, wherein the first chamber section includes a first cavity having a volume from about 0.2 ml to 3 ml.

9. The sperm wash device of claim 1, wherein the second chamber section includes a second cavity having a volume from about 0.2 ml to 3 ml.

10. The sperm wash device of claim 1, wherein the slider has a bottom section attached to a side section, the bottom section configured to cover the slot when the slider is positioned at the second position.

11. The sperm wash device of claim 10, wherein the bottom section is composed of a mesh.

12. The sperm wash device of claim 11, wherein the mesh includes openings having a diameter from about 20 microns to 100 microns.

13. A method for collecting motile sperm with the sperm wash device of claim 1, the method comprising:
a) introducing the unwashed semen sample into the first chamber section while the slider is in the first position;
b) moving the slider to the second position;
c) introducing the sperm wash media into the second chamber section;
d) positioning the slider to the first position;
e) allow sperm to swim from the first chamber section to the second chamber section for a predetermined period of time;
f) positioning the slider in the second position; and
g) collecting the sperm wash media having motile sperm therein.

14. The method of claim 13. wherein the sperm wash device is heated during collection of the motile sperm.

15. The method of claim 14, wherein the sperm wash device is heated to a temperature from about 28 to 45° C. during collection of the motile sperm.

16. The method of claim 14, wherein the predetermined period of time is from about 10 to 90 minutes.

17. The method of claim 14, wherein the predetermined period of time is about 30 minutes.

18. The method of claim 14, wherein the unwashed semen sample is liquified prior to being introduced into the first chamber section.
